(19) United States
(12) Patent Application Publication
(10) Pub. No.: US 2011/0137243 A1
(43) Pub. Date: Jun. 9, 2011

(54) Coating On A Balloon Device

(75) Inventors: Syed F. A. Hossainy, Hayward, CA (US); O. Mikael Trollsas, San Jose, CA (US); Keith R. Cromack, Chesterfield, MO (US); Stephen D. Pacetti, San Jose, CA (US)

(73) Assignee: ABBOTT CARDIOVASCULAR SYSTEMS INC., Santa Clara, CA (US)

(21) Appl. No.: 13/015,377

(22) Filed: Jan. 27, 2011

Related U.S. Application Data
(63) Continuation-in-part of application No. 11/899,792, filed on Sep. 6, 2007.

(51) Int. Cl. A61M 25/10; A61F 2/00; A61K 47/30

(52) U.S. Cl. 604/96.01; 424/423; 514/772.3

(57) ABSTRACT

The present invention provides a hygroscopic coating on a balloon for an implantable device and methods of making and using the same.

### CROSS-REFERENCE TO RELATED APPLICATIONS

This application is a continuation-in-part of U.S. application Ser. No. 11/899,792 filed 6 Sep. 2007, which is incorporated by reference as if fully set forth, including any figures, herein.

### FIELD OF THE INVENTION

The present invention relates to a hygroscopic coating on a balloon of a medical device.

### BACKGROUND OF THE INVENTION

Percutaneous coronary intervention (PCI) is a procedure for treating heart disease. A catheter assembly having a balloon portion is introduced percutaneously into the cardiovascular system of a patient via the brachial or femoral artery. The catheter assembly is advanced through the coronary vasculature until the balloon portion is positioned across the occlusive lesion. Once in position across the lesion, the balloon is inflated to a predetermined size to radially compress the atherosclerotic plaque of the lesion to remodel the lumen wall. The balloon is then deflated to a smaller profile to allow the catheter to be withdrawn from the patient's vasculature.

Problems associated with the above procedure include formation of intimal flaps or torn arterial linings which can collapse and occlude the blood conduit after the balloon is deflated. Moreover, thrombosis and restenosis of the artery may develop over several months after the procedure, which may require another angioplasty procedure or a surgical by-pass operation. To reduce the partial or total occlusion of the artery by the collapse of the arterial lining and to reduce the chance of thrombosis or restenosis, a stent is implanted in the artery to keep the artery open.

Drug delivery stents have reduced the incidence of in-stent restenosis (ISR) after PCI (see, e.g., Serruys, P. W., et al., J. Am. Coll. Cardiol. 39:393-399 (2002)), which has plagued interventional cardiology for more than a decade. However, a few challenges remain in the art of drug delivery stents. One issue is that of late stent thrombosis, where there is a concern that drug delivery stents have a higher incidence than bare metal stents. Another example is the loss of therapeutic agent in the deployment of the stent. Similar problems with loss of therapeutic agent also occur with drug delivery balloons, that is the balloons of a balloon catheter which has a drug coating.

The embodiments of the present invention address the above-identified needs and issues.

### SUMMARY OF THE INVENTION

The present invention provides a coating on a balloon including a hygroscopic coating. The coating includes a hygroscopic layer comprising a hygroscopic polymer(s). In some embodiments, the hygroscopic layer is substantially free from water or moisture and is capable of absorbing water (water absorbing capacity) of about 5 wt % or more of the total weight of the hygroscopic layer water. In some embodiments, the hygroscopic layer can have a water absorbing capacity of about 10 wt % or higher, about 20 wt % or higher, about 40 wt % or higher, about 50 wt % or higher, about 75 wt % or higher, about 90 wt % or higher, about 100 wt % or higher, about 200 wt % or higher, about 400 wt % or higher, about 500 wt % or higher, about 600 wt % or higher, about 700 wt % or higher, about 800 wt % or higher, about 900 wt % or higher, or about 1000 wt % or higher, all by the weight of the hygroscopic layer.

In some embodiments, the hygroscopic layer can have a water absorbing capacity with an upper limit of about 20 wt %, about 40 wt %, about 60 wt %, about 80 wt %, about 100 wt %, about 500 wt %, about 600 wt %, about 700 wt %, about 800 wt %, about 900 wt %, or about 1000 wt %.

In some embodiments, the hygroscopic coating described herein can have a fast water absorbing rate. For example, a hygroscopic coating can absorb water up to a specified amount, measured as a percentage of the dry weight of the hygroscopic coating, within about 1 minute, about 2 minutes, about 5 minutes, about 10 minutes, about 20 minutes, about 30 minutes, or about 60 minutes upon exposure to an aqueous environment. Such specified amount of water absorption, expressed as a percentage of the dry weight of the hygroscopic coating, can be, e.g., about 5 wt %, between about 5 wt % and about 10 wt %, about 10 wt %, between about 10 wt % and 20 wt %, about 20 wt %, between about 20% and 40%, about 40 wt %, between about 40% and about 50%, about 50 wt %, between about 50% and about 75%, about 75 wt %, between about 75 wt % and 90 wt %, about 90 wt %, between about 90 wt % and about 100 wt %, about 100 wt %, between about 100 wt % and about 200 wt %, about 200 wt %, between about 200 wt % and about 400 wt %, about 400 wt %, between about 400 wt % and about 500 wt %, about 500 wt %, between about 500 wt % and about 600 wt %, about 600 wt %, between about 600 wt % and about 700 wt %, about 700 wt %, between about 700 wt % and about 800 wt %, about 800 wt %, between about 800 wt % and about 900 wt %, about 900 wt %, between about 900 wt % and about 1000 wt %, or about 1000 wt %, all by the dry weight of the hygroscopic layer.

In some embodiments, the hygroscopic layer is capable of absorbing between about 20 wt % and about 500 wt % by the dry weight of the hygroscopic layer within about 5 to about 60 minutes upon exposure to an aqueous environment.

In some embodiments, the hygroscopic layer can be formed on a medical device such as a balloon catheter or a drug delivery stent, to minimize drug loss into unwanted areas during the delivery and deployment of the balloon or during stent deployment as a result of its water-absorbing capability to prevent, limit, or mitigate the premature leakage or loss of a drug from the coating into the environment.

In some embodiments, the hygroscopic layer can be formed on a balloon of a balloon catheter or a drug delivery stent for loading a drug in situ by exposing the hygroscopic coating to an aqueous solution of a drug, thereby forming a drug loaded hydrogel coating in situ. The in-situ formation of the drug layer minimizes drug loss that may result from the fabrication, packaging, and/or handling of the device. In these embodiments, the hygroscopic layer can be used to optionally load a second drug or agent. For example, the hygroscopic layer can be included in a coating that has a reservoir layer including a drug or agent in addition to the hygroscopic layer. The hygroscopic layer therefore can be used to load additional drug or agents, which may be the same as or different from the drug(s) or agent(s) than the drug(s) or agent(s) in the reservoir layer.

In addition to a hygroscopic polymer or material described below, the hygroscopic layer can include one or more non-hygroscopic polymers and/or other materials, such as excipients. Such non-hygroscopic polymer can be degradable (bioabsorable) or non-degradable (biostable), which are further described below.

Some exemplary, but non-limiting, bioactive agents that can be included in a coating having a hygroscopic layer described above and/or another layer are paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl (4-amino-TEMPO), biolimus, tacrolimus, dexamethasone, dexamethasone-acetate, rapamycin, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N-1-tetrazolyl)-rapamycin (ABT-578), novolimus, myolimus, temsirolimus, deforolimus, γ-hirudin, clobetasol, pimecrolimus, imatinib mesylate, midostaurin, cRGD, prodrugs thereof, co-drugs thereof, and any combination thereof. Some other examples of the bioactive agents include siRNA and/or other oligoneucleotides that inhibit endothelial cell migration. Another exemplary bioactive agent is lysophosphatidic acid (LPA) or sphingosine-1-phosphate (S1P). LPA is a “bioactive” phospholipid able to generate growth factor-like activities in a wide variety of normal and malignant cell types. LPA plays an important role in normal physiological processes such as wound healing, and in vascular tone, vascular integrity, or reproduction.

The hygroscopic layer can be formed on a medical device such as a balloon of a balloon catheter or a stent. A drug delivery stent can be implanted in a patient to treat, prevent, mitigate, or reduce a medical condition, or to provide a pro-healing effect. Likewise, a balloon including a hygroscopic layer and also including a drug may be inserted into a patient to treat, prevent, mitigate, or reduce a medical condition, or to provide a pro-healing effect. In some embodiments, the medical condition is a coronary artery disease (CAD) or a peripheral vascular disease (PVD). Some examples of such vascular medical diseases are restenosis and/or atherosclerosis. Other conditions that may be treated with a medical device, including those described herein, include, without limitation, thrombosis, hemorrhage, vascular dissection or perforation, vascular aneurysm, vulnerable plaque, chronic total occlusion, claudication, anastomotic proliferation (for vein and artificial grafts), bile duct obstruction, ureter obstruction, tumor obstruction, or combinations of these.

### DETAILED DESCRIPTION

The present invention provides a balloon including a coating including a hydrophilic or hygroscopic polymer in at least one layer (“hygroscopic layer”).

As used herein, the term “substantially free from water” shall mean a water content of less than about 10 wt %, less than about 5 wt %, less than about 1 wt %, less than about 0.1 wt %, or less than about 0.01 wt % water. Water content would be measured at ambient temperature (about 20 to 25° C.) and an atmosphere of dry air, nitrogen, argon, or other inert gas may be required. In some embodiments, with respect to the atmosphere of testing, dry is not more than 1% by mass water, and in other embodiments, dry is not more than 500 ppm (by mass) water.

In some embodiments, the hygroscopic layer is capable of absorbing between about 20 wt % and about 500 wt % of its dry weight within about 5 to 60 minutes upon exposure to an aqueous environment.

As used herein, the term “absorb” is used interchangeably with the term “swell.”

In some embodiments, the hygroscopic layer can be formed on a drug coated balloon of a balloon catheter, or on a drug delivery stent, to minimize drug loss into unwanted areas during delivery for a balloon or delivery and deployment of the stent. For balloon coatings, particularly those including a drug, the coating typically comes off the balloon during deployment and during the treatment procedure. The duration of the treatment procedure is typically about 1 to about 5 minutes. Some coating may also come off the balloon during the delivery. In contrast, a drug coating on a stent is typically intended to remain on the stent. Loss of coating from a stent during delivery and deployment is undesirable.

In some embodiments, the hygroscopic layer is frangible such that upon expansion of the balloon the hygroscopic layer breaks so as to allow the bioactive agent in the drug layer to be quickly released into the aqueous environment. As used herein, the term “quickly release” refers to a release rate of up to about 50% to about 100% of the agent or drug from the drug layer or reservoir layer on the balloon within a period of about 5 minutes to about 60 minutes.

In some embodiments, about 50% to about 100% of the drug is released in less than 5 minutes. In other embodiments, about 50% to about 100% of the drug is released in about 4 minutes or less than 4 minutes, and in still other embodiments, in about 2 minutes or less than 2 minutes.

Embodiments of the present invention encompass hygroscopic layers that dissolve (at least 80% by weight of the layer) in a relatively short time. The upper limit for a relatively short time may be about 10 minutes, about 8 minutes, or about 5 minutes. The lower limit for a short duration of time may be about 30 seconds, about 1 minute, or about 2 minutes. Embodiments of the present invention encompass all durations of time resulting from combining one of the above upper limits with one of the above lower limits. As used herein, dissolution time encompasses not only true dissolution of the coating, that is when the components of the layer are dissolved forming a true solution, but also encompasses the transfer of the coating to the vessel wall followed by coating dissolution. In some embodiments, the time frame for measuring the “dissolution” of the layer begins at the time of the introduction of the device into a patient. In other embodiments, the time frame for measuring “dissolution” of the layer begins at the time of deployment, that is the expansion of the device.

In addition to a hygroscopic polymer described below, the hygroscopic layer can include a non-hygroscopic polymer and/or another material, such as an excipient. Such non-hygroscopic polymer can be degradable (bioabsorbable) or non-degradable (biostable), which is further described below.

### Definitions

Wherever applicable, the definitions provided below shall apply for the terms used throughout the description of the present invention.

Use of the singular herein includes the plural and vice versa unless expressly stated to be otherwise. That is, “a” and “the” refer to one or more of whatever the word modifies. For example, “a drug” may refer to one drug, two drugs, etc. Likewise, “the balloon” may refer to one, two or more balloons and “the polymer” may mean one polymer or a plurality of polymers. By the same token, words such as, without limitation, “balloons” and “polymers” would refer to one balloon or polymer as well as to a plurality of balloons or polymers unless it is expressly stated or obvious from the context that such is not intended.

As used herein, words of approximation such as, without limitation, “about,” “substantially,” “essentially,” and “approximately” mean that the word or phrase modified by the term need not be exactly that which is written but may vary from that written description to some extent. The extent to which the description may vary will depend on how great a change can be instituted and have one of ordinary skill in the art recognize the modified version as still having the properties, characteristics and capabilities of the modified word or phrase. In general, but with the preceding discussion in mind, a numerical value herein that is modified by a word of approximation may vary from the stated value by ±15%, unless expressly stated otherwise.

The terms “biodegradable,” “bioerodable,” “bioabsorbable,” “bioresorbable,” and “degradable,” in reference to polymers, materials, and coatings, are used interchangeably and refer to polymers, materials, and coatings that are capable of being completely or substantially completely degraded, dissolved, and/or eroded over time when exposed to physiological conditions, and can be gradually resorbed, absorbed and/or eliminated by the body, or that can be degraded into fragments that can pass through the kidney membrane of an animal (e.g., a human), e.g., fragments having a molecular weight of about 40,000 Daltons (40 kDa) or less. The process of breaking down and eventual absorption and elimination of the polymer, material, or coating can be caused by, e.g., hydrolysis, metabolic processes, oxidation, enzymatic processes, bulk or surface erosion, and the like. Conversely, a “biostable” polymer or coating refers to a polymer or coating that is not biodegradable, or is non-degradable.

Whenever the reference is made to “biodegradable,” “bioerodable,” “bioabsorbable,” “bioresorbable,” and “degradable” coatings, materials and/or polymers used as coatings, it is understood that after the process of degradation, erosion, absorption, and/or resorption has been completed or substantially completed, no coating, material, or polymer, or substantially little coating, material, or polymer, will remain on the device.

“Physiological conditions” refer to conditions to which an implant is exposed within the body of an animal (e.g., a human). Physiological conditions include, but are not limited to, “normal” body temperature for that species of animal (approximately 37° C. for a human) and an aqueous environment of physiologic ionic strength, pH and enzymes. In some cases, the body temperature of a particular animal may be above or below what would be considered “normal” body temperature for that species of animal. For example, the body temperature of a human may be above or below approximately 37° C. in certain cases. The scope of the present invention encompasses such cases where the physiological conditions (e.g., body temperature) of an animal are not considered “normal.”

As used herein, “solvent” refers to a substance capable of dissolving one or more other substances or capable of at least partially dissolving the other substance(s) to form a uniformly dispersed solution at the molecular- or ionic level at a selected temperature and pressure. A solvent can refer to one chemical compound, or a mixture of chemical compounds. A solvent can be a fluid. A substance may be a solvent even though the amount of another substance that it can dissolve is very small.

As used herein, “therapeutic agent,” “drug,” “active agent,” “bioactive agent,” “biologically active agent,” and “biological agent” are used interchangeably, and refer to any substance that, when administered in a therapeutically effective amount to a patient (an animal, including a human being) suffering from a disease or condition, has a therapeutic beneficial effect on the health and well-being of the patient. A therapeutic beneficial effect on the health and well-being of a patient includes, but is not limited to: (1) curing the disease or condition; (2) slowing the progress of the disease or condition; (3) causing the disease or condition to retrogress; or, (4) alleviating one or more symptoms of the disease or condition.

As used herein, a drug also includes any substance that when administered to a patient, known or suspected of being particularly susceptible to a disease, in a prophylactically effective amount, has a prophylactic beneficial effect on the health and well-being of the patient. A prophylactic beneficial effect on the health and well-being of a patient includes, but is not limited to: (1) preventing or delaying on-set of the disease or condition in the first place; (2) maintaining a disease or condition at a retrogressed level once such level has been achieved by a therapeutically effective amount of a substance, which may be the same as or different from the substance used in a prophylactically effective amount; or, (3) preventing or delaying recurrence of the disease or condition after a course of treatment with a therapeutically effective amount of a substance, which may be the same as or different from the substance used in a prophylactically effective amount, has concluded.

As used herein, “drug” also refers to pharmaceutically acceptable, pharmacologically active derivatives of those drugs specifically mentioned herein, including, but not limited to, salts, esters, amides, prodrugs, active metabolites, analogs, and the like. As used herein, “drug” also refers to diagnostic agents.

In the context of a blood-contacting implantable or insertable medical device, a “prohealing” drug or agent refers to a drug or agent that has the property that it promotes or enhances re-endothelialization of arterial lumen to promote healing of the vascular tissue.

As used herein, a “co-drug” is a drug that is administered concurrently or sequentially with another drug to achieve a particular pharmacological effect. The effect may be general or specific. The co-drug may exert an effect different from that of the other drug, or it may promote, enhance or potentiate the effect of the other drug.

As used herein, the term “prodrug” refers to an agent rendered less active by a chemical or biological moiety, which metabolizes into or undergoes in vivo hydrolysis to form a drug or an active ingredient thereof. The term “prodrug” can be used interchangeably with terms such as “proagent,” “latentiated drugs,” “bioreversible derivatives,” and “congeners.” N. J. Harper, Drug latentiation, Prog Drug Res., 4: 221-294 (1962); E. B. Roche, Design of Biopharmaceutical Properties through Prodrugs and Analogs, Washington, D.C.: American Pharmaceutical Association (1977); A. A. Sinkula and S. H. Yalkowsky, Rationale for design of biologically reversible drug derivatives: prodrugs, J. Pharm. Sci., 64: 181-210 (1975). Use of the term “prodrug” usually implies a covalent link between a drug and a chemical moiety, though some authors also use it to characterize some forms of salts of the active drug molecule. Although there is no strict universal definition of a prodrug itself, and the definition may vary from author to author, prodrugs can generally be defined as pharmacologically less active chemical derivatives that can be converted in vivo, enzymatically or nonenzymatically, to the active, or more active, drug molecules that exert a therapeutic, prophylactic or diagnostic effect. Sinkula and Yalkowsky, above; V. J. Stella et al., Prodrugs: Do they have advantages in clinical practice?, Drugs, 29: 455-473 (1985).

The terms “polymer” and “polymeric” refer to compounds that are the product of a polymerization reaction. A “polymer” also refers to a molecule comprised of repeating “constitutional units.” The constitutional units may derive from the reaction of monomers. As a non-limiting example, ethylene (CH₂═CH₂) is a monomer that can be polymerized to form polyethylene, CH₃CH₂(CH₂CH₂)—CH₂CH₃ (where n is an integer), wherein the constitutional unit is —CH₂CH₂—, ethylene having lost the double bond as the result of the polymerization reaction. The constitutional units themselves can be the product of the reactions of other compounds. The terms “polymer” and “polymeric” are inclusive of homopolymers (i.e., polymers obtained by polymerizing one type of monomer), copolymers (i.e., polymers obtained by polymerizing two or more different types of monomers), terpolymers (a subset of copolymers), etc., including random, alternating, block, graft, linear, branched, dendritic, star, comb, and any other variations thereof. Polymers may also be cross-linked to form a network. As used herein, the term polymer refers to a molecule comprising more than 20 constitutional units.

As used herein, a molecule which has a chain length of 20 or fewer constitutional units is referred to as an “oligomer.”

The glass transition temperatures, T_{g}, is the temperature at which a material such as a polymer (or a segment of a polymer) which is in an amorphous phase changes mechanical properties from those of a rubber (i.e., elastic) to those of a glass (brittle). Although materials other than polymers may also have a T_{g} if the material is supercooled without crystallizing, the term is most often used when referring to polymers. The measured T_{g} of a polymer (or polymer segment) may vary depending on the method used to determine it. One such method is differential scanning calorimetry, which is the method employed herein. The measurement of T_{g} using differential scanning calorimety is influenced by the rate of heating or cooling.

Plasticization of a polymer, refers to the addition of a lower molecular weight material to the polymer. The effect is to lower the T_{g} of the blend of the polymer and platiscizer, to thereby transform a hard, brittle polymer to a soft, rubber-like polymer. The uptake of a plasticizer allows for greater polymer chain mobility, and as a result, a lower T_{g}.

As used herein, the term “implantable” refers to the attribute of being implantable in a mammal (such as a human being), that is it may be any suitable substrate that can be implanted in a human or non-human animal and that meets the mechanical, physical, chemical, biological, and pharmacological requirements of a device provided by laws and regulations of a governmental agency (e.g., the U.S. FDA) such that the device is safe and effective for use as indicated by the device. Examples of implantable devices include, but are not limited to, self-expandable stents, balloon-expandable stents, coronary stents, peripheral stents, stent-grafts, catheters, other expandable tubular devices for various bodily lumen or orifices, grafts, vascular grafts, arterio-venous grafts, by-pass grafts, pacemakers and defibrillators, leads and electrodes for the preceding, artificial heart valves, anastomotic clips, arterial closure devices, patent foramen ovale closure devices, cerebrospinal fluid shunts, and particles (e.g., drug-eluting particles, microparticles and nanoparticles).

Other medical devices may be referred to as insertable medical devices, that is any type of appliance that is totally or partly introduced, surgically or medically, into a patient's body or by medical intervention into a natural orifice, but the device does not remain in the patient's body after the procedure. As used herein, insertable medical devices are a subset of implantable devices in which the duration of the implantation is for a limited time period.

Another type of medical device is a vascular catheter. Vascular catheters are insertable medical devices that are removed after the procedure. A vascular catheter is a thin, flexible tube with a manipulating means at one end, which remains outside the patient's body, and an operative device at or near the other end, which is inserted into the patient's artery or vein. The catheter may be introduced into a patient's vasculature at a point remote from the target site, e.g., into the femoral artery where the target is the heart. The catheter is steered, assisted by a guide wire than extends through a lumen in the flexible tube, to the target site whereupon the guide wire is withdrawn at which time the lumen may be used for the introduction of fluids, often containing drugs, to the target site. A catheter may also be used to deliver a stent or may be used to deliver a balloon used in angioplasty.

As used herein, a “balloon” refers to the well-known in the art device, usually associated with a vascular catheter, that comprises a relatively thin, flexible material, forming a tubular membrane, that when positioned at a particular location in a patient's vessel can be expanded or inflated to an outside diameter that is essentially the same as the inside or luminal diameter of the vessel in which it is placed. In addition to diameter, a balloon has other dimensions suitable for the vessel in which it is to be expanded. Balloons may be inflated using a liquid medium, such as but without limitation, water or normal saline solution, that is, saline that is essentially isotonic with blood.

A “balloon catheter” refers to a catheter with a balloon attached to the tube near its distal end.

Stents, catheters, and catheter balloons may be used in any vessel in the body, including neurological, carotid, vein graft, coronary, aortic, renal, iliac, femoral, popliteal vasculature, and urethral passages.

As used herein, a material that is described as a layer or a film (a coating) “disposed over” an indicated substrate (such as a medical device) refers to a coating of the material deposited directly or indirectly over at least a portion of the surface of the substrate. Direct depositing means that the coating is applied directly to the exposed surface of the substrate. Indirect depositing means that the coating is applied to an intervening layer that has been deposited directly or indirectly over the substrate. A coating layer is supported by a surface of the substrate, whether the coating layer is deposited directly, or indirectly, onto the surface of the substrate. A coating may include multiple layers or may be only one layer. As used herein, unless the context indicates otherwise or it is expressly stated otherwise, a coating layer is not chemically or covalently bound to the substrate, or to a another coating layer onto which it has been deposited.

### Hygroscopic Polymers

Any polymer that has a water absorbing capacity can be used to form a hygroscopic layer of this invention. As used herein, the term “hygroscopic polymer” refers to an oligomer as well as a polymer per se of the same constitutional units. The layer may include only a hygroscopic polymer, a hygroscopic polymer and another polymer, or a hygroscopic polymer, a non-hygroscopic polymer, and another material.

In some embodiments, the hygroscopic polymer may be a hydrophilic polymer, such as and without limitation, polyvinylpyrrolidone (PVP), poly(carboxymethyl cellulose) (poly(CMC)), poly(ethylene glycol) (PEG), and poly[N-(2-hydroxypropyl)methacrylamide] (poly(HPMA)).

In some embodiments, the hygroscopic polymer can be an amphiphilic polymer. In some further embodiments, the hygroscopic polymer can be a hydrophobic polymer with hygroscopic moieties or pendant groups covalently attached to the hydrophobic polymer. Examples of such hygroscopic moieties or pendant groups include, without limitation, choline, phosphoryl choline, ammonium groups, carboxyl, phosphate, and sulfonic acid groups. Examples of amphiphilic polymers include, but are not limited to, poly(n-butyl methacrylate-phosphorylcholine) (PBMA-PC), poly(ester amide)-phosphorylcholine (PEA-PC), polylactide-phosphorylcholine (PLA-PC), polylactide-phosphorylcholine (PLA-PC), polyethylene glycol-poly(caprolactone)-di- or tri-blocks (PEG-PCL), polyethylene glycol-polylactide di- or tri-blocks (PEG-PLA), polyethylene glycol-poly(lactide-glycolide) di- or tri-blocks (PEG-PLGA), PLURONIC® (poly(ethylene oxide)-b-poly(propylene oxide)-b-poly(ethylene oxide)) etc. The term “poloxamer” refers to tri-block copolymers with a central block of polypropylene oxide) (PPO) and with a block of poly(ethylene oxide) (PEO) on each side where the PEO blocks are usually of the same length in terms of number of constitutional units. Poloxamers of types 124, 188, 237, 338, and 407 are specified by a monograph in the National Formulary. Some PLURONIC® polymers sold by BASF also meet one of the NF specifications for a type of poloxamer.

In some further embodiments, the hygroscopic polymer can be a dissolvable polymer. As used herein, the term dissolvable polymer refers to a polymer that can be dissolved in water or a predominantly aqueous environment. Examples of dissolvable polymers include, without limitation: polyvinyl alcohol (PVA); poly n-butyl methacrylate-phosphorylcholine (PBMA-PC); poly(ester amide)-phosphorylcholine (PEA-PC); polylactide-phosphorylcholine (PLA-PC); poly(lactic acid-co-glycolic acid)-phosphorylcholine (PLGA-PC); silk-elastin, dynamers, or tri-blocks such as poly(caprolactone)-polyethylene glycol-poly(caprolactone) (PCL-PEG-PCL); (PLA-PEG-PLA); and poly(lactide-glycolide)-polyethylene glycol-poly(lactide-glycolide) tri-blocks (PLGA-PEG-PLGA).

In some embodiments, the hygroscopic polymer is a poly(ester-amide), that is a polymer that has in its backbone structure both ester and amide bonds.

For balloon coatings, particularly those including a drug, the coating typically comes off the balloon during the deployment and during the treatment which is typically of a duration of about 1 to about 5 minutes. Some of the coating may also come off during the delivery to the treatment site. In contrast, a drug coating on a stent is typically intended to remain on the stent. The hygroscopic polymer is one which will be released in the blood stream if used as a coating on a balloon of a vascular balloon catheter. Because the materials are released into the blood stream, the materials are excreted from the body via the kidneys. There is a limit on the molecular weight of materials that can pass through the kidney. For those embodiments involving a coating on a balloon of a vascular balloon catheter, the number average molecular weight of the polymer is not greater than 40,000 Daltons, and preferably not greater than 35,000 Daltons. In various embodiments, the lower limit of the number average molecular weight may be not less than 1000 Daltons, preferably not less than 3000 Daltons, and even more preferably not less than 5000 Daltons.

As discussed above, some embodiments encompass hygroscopic layers that dissolve (at least 80% by weight of the layer) in a relatively short duration of time. Thus, the hygroscopic polymer may have a number average molecular weight of not less than 500 and not more than 20,000 Daltons, preferably not less than 1000 and not more than 15,000 Daltons, and even more preferably not less than 1500 Daltons and not more than 12,000 Daltons.

The hygroscopic polymers are preferably uncross-linked, particularly if used as part of a hygroscopic coating for a balloon of a balloon catheter

In those embodiments involving the coating of another type of implantable medical device such as a stent or a catheter shaft, the polymers may be of a higher molecular weight than 40,000 Daltons (number average molecular weight). In some embodiments involving the coating of another type of implantable medical device, particularly if coating on a catheter shaft (flexible tube), the polymers are preferably cross-linked and/or covalently attached to the substrate.

### Biologically Active Agents

The coating on the balloon or other implantable device described herein can optionally include at least one biologically active (“bioactive”) agent. A bioactive agent may be included in the hygroscopic layer, and/or in another layer (the “drug layer” or “reservoir layer”). In some embodiments, the body of a device or a portion of the device may include a bioactive agent.

Examples of suitable bioactive agents include, but are not limited to, synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules that bind to complementary DNA to inhibit transcription, and ribozymes. Other examples of bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. The bioactive agents could be designed, e.g., to inhibit the activity of vascular smooth muscle cells, or they could be directed at inhibiting abnormal or inappropriate migration and/or proliferation of smooth muscle cells to inhibit restenosis.

In certain embodiments, optionally in combination with one or more other embodiments described herein, the device can include at least one biologically active agent selected from antiproliferative, antineoplastic, antimitotic, anti-inflammatory, cystostatic, antiplatelet, anticoagulant, antifibrin, antithrombin, antibiotic, antiallergic and antioxidant substances.

An antiproliferative agent can be a natural proteineous agent such as a cytotoxin or a synthetic molecule. Examples of antiproliferative substances include, but are not limited to, actinomycin D or derivatives and analogs thereof (manufactured by Sigma-Aldrich, or COSMEGEN™ available from Merck) (synonyms of actinomycin D include dactinomycin, actinomycin IV, actinomycin I₁, actinomycin X₁, and actinomycin C₁); all taxoids such as taxols, docetaxel, and paclitaxel and derivatives thereof all “mTOR” drugs including, without limitation, sirolimus (rapamycin), biolimus A9 (Biosensors International, Singapore), deforolimus, AP23572 (Ariad Pharmaceuticals), tacrolimus, temsirolimus, pimecrolimus, novolimus, myolimus, zotarolimus (ABT-578, CAS # 221877-54-9), 40-O-(2-hydroxy)ethyl-rapamycin (everolimus), 40-O-(3-hydroxypropyl)rapamycin, 40-O-[2-(2-hydroxy)ethoxy]ethyl-rapamycin, 40-O-tetrazole-rapamycin, 40-O-tetrazolylrapamycin, 40-epi-(N-1-tetrazole)-rapamycin, and other bioactive agents include pirfenidone, macrolide antibiotics, FKBP-12 mediated mTOR inhibitors, prodrugs thereof, co-drugs thereof, and combinations thereof. In addition, the bioactive agent may be a compound having the structure of rapamycin but with a substituent at the carbon corresponding to the 42 or 40 carbon. The 42 or 40 carbon (“C42/C40” below) does not refer to two different carbon atoms located on the rapamycin molecule, but to the same carbon atom, as illustrated below, with a different label depending upon the numbering scheme used.

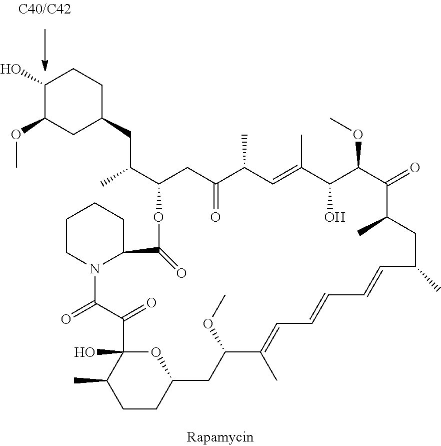

An anti-inflammatory drug or agent can be a steroidal anti-inflammatory drug, a nonsteroidal anti-inflammatory drug (NSAID), or a combination thereof. Examples of anti-inflammatory drugs include, but are not limited to, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol, clobetasol propionate, clobetasone butyrate, clopirac, cloticasone propionate, cormethasone acetate, cortodoxone, deflazacort, desonide, desoximetasone, dexamethasone, dexamethasone acetate, dexamethasone dipropionate, dexamethasone phosphate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, prodrugs thereof, co-drugs thereof, and combinations thereof.

Alternatively, the anti-inflammatory agent can be a biological inhibitor of pro-inflammatory signaling molecules. Anti-inflammatory biological agents include antibodies to such biological inflammatory signaling molecules.

Examples of antineoplastics and/or antimitotics include, but are not limited to, paclitaxel (e.g., TAXOL® available from Bristol-Myers Squibb), docetaxel (e.g., TAXOTERE® from Aventis), methotrexate, azathioprine, vincristine, vinblastine, fluorouracil, doxorubicin hydrochloride (e.g., ADRIAMYCIN® from Pfizer), and mitomycin (e.g., MUTAMYCIN® from Bristol-Myers Squibb).

Examples of antiplatelet, anticoagulant, antifibrin, and antithrombin agents that can also have cytostatic or antiproliferative properties include, but are not limited to, sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as ANGIOMAX™ (from Biogen), calcium channel blockers (e.g., nifedipine), colchicine, fibroblast growth factor (FGF) antagonists, fish oil (e.g., omega 3-fatty acid), histamine antagonists, lovastatin (a cholesterol-lowering drug that inhibits HMG-CoA reductase, brand name MEVACOR® from Merck), monoclonal antibodies (e.g., those specific for platelet-derived growth factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetics, 4-amino-2,2,6,6-tetramethylpiperidine-1-oxyl (4-amino-TEMPO), estradiol, anticancer agents, dietary supplements such as various vitamins, and a combination thereof.

Examples of cytostatic substances include, but are not limited to, angiopeptin, angiotensin converting enzyme inhibitors such as captopril (e.g., CAPOTEN® and CAPOZIDE® from Bristol-Myers Squibb), cilazapril and lisinopril (e.g., PRINIVIL® and PRINZIDE® from Merck).

Examples of antiallergic agents include, but are not limited to, permirolast potassium. Examples of antioxidant substances include, but are not limited to, 4-amino-2,2,6,6-tetramethylpiperidine-1-oxyl (4-amino-TEMPO).

Other bioactive agents include anti-infectives such as antiviral agents; analgesics and analgesic combinations; antihelmintics; antiarthritics, antiasthmatic agents; anticonvulsants; antidepressants; antidiuretic agents; antidiarrheals; antihistamines; antimigrain preparations; antinauseants; antiparkinsonism drugs; antipruritics; antipsychotics; antipyretics; antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary vasodilators; peripheral and cerebral vasodilators; central nervous system stimulants; hypnotics; immunosuppressives; muscle relaxants; parasympatholytics; sedatives; tranquilizers; naturally derived or genetically engineered lipoproteins; and restenoic reducing agents.

Other biologically active agents that can be used include alpha-interferon, and genetically engineered epithelial cells.

In some embodiments, the bioactive agent is a “prohealing” drug or agent. A “prohealing” drug or agent, in the context of a blood-contacting implantable device, refers to a drug or agent that has the property that it promotes or enhances re-endothelialization of arterial lumen to promote healing of the vascular tissue. The portion(s) of an implantable device containing a prohealing drug or agent can attract, bind and eventually become encapsulated by endothelial cells (e.g., endothelial progenitor cells.

The prohealing drug or agent can be dispersed in the body of the hygroscopic polymer substrate or scaffolding. The prohealing drug or agent can also be dispersed, uniformly or non-uniformly, within a hygroscopic coating over a surface of an implantable device, such as a stent or a balloon catheter.

“Endothelial progenitor cells” refer to primitive cells made in the bone marrow that can enter the bloodstream and go to areas of blood vessel injury to help repair the damage. Endothelial progenitor cells circulate in adult human peripheral blood and are mobilized from bone marrow by cytokines, growth factors, and ischemic conditions. Vascular injury is repaired by both angiogenesis and vasculogenesis mechanisms. Circulating endothelial progenitor cells contribute to repair of injured blood vessels mainly via a vasculogenesis mechanism.

In some embodiments, the prohealing drug or agent can be an endothelial cell (EDC)-binding agent. In certain embodiments, the EDC-binding agent can be a protein, peptide or antibody, which can be, e.g., one of collagen type 1, a 23 peptide fragment known as single chain Fv fragment (scFv A5), a junction membrane protein vascular endothelial (VE)-cadherin, and combinations thereof. Collagen type 1, when bound to osteopontin, has been shown to promote adhesion of endothelial cells and modulate their viability by the down regulation of apoptotic pathways. Endothelial cells can be selectively targeted (for the targeted delivery of immunoliposomes) using scFv A5. Junction membrane protein vascular endothelial (VE)-cadherin has been shown to bind to endothelial cells and down regulate apoptosis of the endothelial cells.

In a particular embodiment, the EDC-binding agent can be the active fragment of osteopontin, (Asp-Val-Asp-Val-Pro-Asp-Gly-Asp-Ser-Leu-Ala-Try-Gly). Other EDC-binding agents include, but are not limited to, EPC (epithelial cell) antibodies, RGD peptide sequences, RGD mimetics, and combinations thereof.

In further embodiments, the prohealing drug or agent can be a substance or agent that attracts and binds endothelial progenitor cells. Representative substances or agents that attract and bind endothelial progenitor cells include antibodies such as CD-34, CD-133 and vegf type 2 receptor. An agent that attracts and binds endothelial progenitor cells can include a polymer having nitric oxide donor groups.

The foregoing biologically active agents are listed by way of example and are not meant to be limiting. Other biologically active agents that are currently available or that may be developed in the future are equally applicable.

Any of the above listed bioactive agents may be used in combination with any one or more of any of the other above listed bioactive agents.

In a more specific embodiment, optionally in combination with one or more other embodiments described herein, the coating of the invention comprises at least one biologically active agent selected from paclitaxel, docetaxel, estradiol, nitric oxide donors, super oxide dismutases, super oxide dismutase mimics, 4-amino-2,2,6,6-tetramethylpiperidine-1-oxyl (4-amino-TEMPO), tacrolimus, dexamethasone, dexamethsone-acetate, rapamycin, compounds having the structure of rapamycin but with a substituent at the carbon corresponding to the 42 or 40 carbon, everolimus, biolimus, 40-O-(3-hydroxy)propyl-rapamycin, 40-O-[2-(2-hydroxy)ethoxy]ethyl-rapamycin, 40-O-tetrazole-rapamycin, zotarolimus, novolimus, myolimus, pimecrolimus, imatinib mesylate, midostaurin, clobetasol, progenitor cell-capturing antibodies, prohealing drugs, cRGD, prodrugs thereof, co-drugs thereof, and any combination thereof. In a particular embodiment, the bioactive agent is everolimus or zotarolimus. In another specific embodiment, the bioactive agent is dexamethasone.

### Coating Construct

According to some embodiments of the invention, optionally in combination with one or more other embodiments described herein, a coating disposed over an implantable medical device such as a balloon of a balloon catheter, and/or over a stent, can be a multi-layer structure or a single layer. In some embodiments, the coating includes only the hygroscopic layer which may include a bioactive agent. In some embodiments, the coating includes at least one reservoir layer and a hygroscopic layer, which are layer (2) and layer (3) described below, and can include layer (1) as described below:

(1) a primer layer;

(2) a reservoir layer which can be a drug-polymer layer including at least one polymer or, alternatively, a polymer-free drug layer; and

(3) a hygroscopic layer.

The term “reservoir layer,” as used herein, will be used interchangeably with the term “drug layer.”

In some embodiments, the coating further includes one or more other layers not specifically recited above. Coatings may include more than one reservoir layer and/or more than one hygroscopic layer. In preferred embodiments, the outermost layer is a hygroscopic layer, but any one or more layers or any combination of layers may include a hygroscopic polymer.

The primer layer can be formed of any polymer or material suitable for an implantable medical device, which can be a bioabsorbable, biostable polymer, or a combination thereof.

The reservoir layer or drug layer can include a bioactive agent with or without a hygroscopic polymer described herein.

For a coating on a balloon of a balloon catheter, the drug reservoir layer may have a drug loading between 10 to 1000 ug/cm², and preferably 50 to 750 ug/cm². In some embodiments, the drug loading may be in the range from 100 to 600 ug/cm², and in other embodiments, in the range of 150 to 600 ug/cm². In still other embodiments, the drug loading in the coating layer may be from 250 to 550 ug/cm², or more narrowly from 300 to 500 ug/cm². Embodiments of the present invention also encompass a coating with a drug loading in any of the above drug loading ranges. In contrast, for a drug layer for a coating on a stent, the drug loading may be 10 to 600 ug/cm², and preferably from 50 to 200 ug/cm².

The drug loading as a weight % of the drug layer may be about 40% to about 100%, preferably about 50% to about 99%, and more preferably about 55% to about 98% for a coating on a balloon catheter. In some embodiments, the drug layer may be not less than 85% drug.

In some embodiments, layer (2) and layer (3) can optionally include a bioabsorbable polymer, a biostable polymer, or a combination of a biostable and a bioabsorbable polymer.

Any of the above layers including the hygroscopic layer, the drug layer, and the primer layer, may also include other materials. In some embodiments, the primer layer includes only a polymer, the drug layer includes only a polymer and a drug, and/or the hygroscopic layer includes only a polymer. Any of the layers may also include other excipients, such as, but not limited to, surfactants, binders, fillers, lubricants, plasticizers, anti-static agents, anti-tack agents, anti-foaming agents, stabilizers, anti-oxidants, and/or other additives for pH adjustment.

As discussed above, the hygroscopic layer can have a water absorbing capacity of about 10 wt % or higher, up to about 1000 wt % or higher, all by weight of the hygroscopic layer. The degree of swelling in a hygroscopic layer is controlled by the choice of hygroscopic polymer for the layer, the weight percent of the hygroscopic layer which is the hygroscopic polymer, and to some extent, and the number average molecular weight of the hygroscopic polymer. The upper limit of the weight % of the hygroscopic polymer in the hygroscopic layer may be 100%, 90%, or 80%, and the lower limit may be 30%, 50%, or 60%. Embodiments of the present invention encompass all ranges resulting from combining any one of the above upper limits with any one of the above lower limits of the weight % of the hygroscopic polymer or material in the hygroscopic layer.

Also, as discussed above, in some embodiments, the hygroscopic layer is capable of absorbing between about 20 wt % and about 500 wt % by the dry weight of the hygroscopic layer within about 5 to 60 minutes upon exposure to an aqueous environment. The rate of swelling depends upon not only the type and weight % of the hygroscopic polymer in the hygroscopic layer, but also the thickness of the layer. In some embodiments the hygroscopic layer is about 1 to 50 microns in thickness, preferably about 2 to 25 microns in thickness, and even more preferably, about 4 to 20 microns in thickness. In some embodiments, the thickness of the hygroscopic layer, before swelling, is about 25% to about 100% of the thickness of the drug layer. The presence of lower molecular weight species, a non-limiting example of which is salt, may enhance water intrusion into the layer, particularly if these materials create an osmotic driving force for water absorption. Plasticizers may also enhance the rate of swelling by increasing the diffusion coefficient of water into the hygroscopic layer. In particular, the hygroscopic polymer may be blended with a plasticizer. Examples of plasticizers include without limitation glycerol, propylene glycol, liquid PEG (MW<500 Daltons), liquid polypropylene glycol), N-methylpyrrolidone, dimethylsulfoxide, benzyl alcohol, citrate esters, TWEEN™ 20 (polysorbate 20), and TWEEN™ 80 (polysorbate 80). Polysorbates are a group of oleate esters of sorbitol and its' anhydrides condensed with polymers of ethylene oxide. Polysorbates are used as emulsifiers and surfactants in food, pharmaceuticals and cosmetics. Examples include polysorbate 20, polysorbate 60, and polysorbate 80 the specifications of which are all listed in the USP.

The weight percent plasticizer in the layer may be 0.1 to 50%, preferably 1 to 25%, and even more preferably 2 to 20%. In some embodiments, the weight ratio of plasticizer to hygroscopic polymer or material is between about 1/100 and 1/1, preferably from 2/100 to 40/100, and even more preferably from 4/100 to 20/100. In some embodiments, the hygroscopic layer consists essentially of the hygroscopic polymer, and a plasticizer.

In some embodiments the coating may be free of substances having a molecular weight of greater than 40,000 Daltons, preferably free of substances greater than 35,000 Daltons, and even more preferably, free of substances greater than 30,000 Daltons. For polymers and oligomers, the molecular weight referenced in the above sentence is the number-average molecular weight.

In a preferred embodiment, there is a drug layer and a hygroscopic layer, the hygroscopic layer including poly(vinyl pyrrolidone), and one or more plasticizers selected from the group consisting of glycerol, propylene glycol, liquid PEG (MW<500 Daltons), liquid poly(propylene glycol), N-methylpyrrolidone, dimethylsulfoxide, benzyl alcohol, citrate esters, TWEEN™ 20 (polysorbate 20), and TWEEN™ 80.

Materials from one layer may incidentally migrate from one layer to one or more other layers in the coating during or after application to the substrate. Embodiments of the present invention cover devices with coatings, and coatings themselves, formed by the application of a series of layers as described above. As a non-limiting example, embodiments of the present invention encompass a device with a coating formed by the application of a primer layer, the application of a drug layer above the primer layer, and the application of a hygroscopic layer above the drug layer where the various layers may include the compositions as described above. Embodiments also encompass coated devices and coatings obtained by the application of one or more intervening layers in addition to or instead of those described above.

### Method of Fabricating Implantable Device

Other embodiments of the invention, optionally in combination with one or more other embodiments described herein, are drawn to a method of fabricating an implantable device comprising a balloon. In one embodiment, the method comprises forming a hygroscopic layer or coating on the balloon.

Coating applications typically involve dissolving and/or dispersing the materials used for forming a coating layer, that is polymer, optional drug, and any optional excipient, in a solvent to form a coating solution. The coating solution may be deposited directly onto the surface of the substrate, or the coating solution may be deposited onto a pre-existing coating on the surface of the substrate. For a stent, an example of the substrate surface would be the surface of the “device body,” that is the functional device without a coating or layer of material different from that of which the device body is manufactured has been applied. For a balloon, the substrate surface would be the surface of the balloon measured when inflated to a nominal pressure.

The coating solution may be disposed over all of the outer surface of the device, or a portion of the outer surface of the device. The coating may be continuous or discontinuous (uncoated patches or “holes” may exist). For a medical device which is inserted into the body, “outer surface” is meant any surface however spatially oriented that is in contact with bodily tissue or fluids.

In some embodiments, the coating may be deposited on selective portions of the outer surface. As a non-limiting example, for a stent the coating may be selectively formed on the abluminal surface, that is the surface in contact with the vessel walls. Another non-limiting example is selectively coating the cylindrical surface corresponding to the working length of a balloon of a balloon catheter, that is the surface area of the balloon that would contact the lumen wall when inflated. As used herein, unless expressly stated otherwise, the phrases “coating a catheter balloon,” or a “coated catheter balloon,” will refer to the process of coating, or a coating formed on, the entire surface, or a portion of the surface, of the balloon of the balloon catheter, but not including the catheter shaft. With respect to other implantable medical devices, unless specified otherwise, a reference to the process of coating the device or a coated device will refer to a coating that covers all of, or substantially all of, the outer surface of the device. In a preferred embodiment, for a hygroscopic coating on a balloon, the coating is selectively disposed over the cylindrical surface corresponding to the working length of the balloon.

The coating solution may be disposed over the surface of the substrate by procedures such as spraying the solution onto the substrate, immersing or dipping the substrate in the solution, dripping the solution onto the surface, brushing or wiping the surface with the solution, rolling the device in the solution, ink jet application of the solution, silk screening, or direct fluid dispensing or pipetting of the solution onto the surface. Such coating procedures are well-known in the art. If spraying is used as an application method, the solution may be atomized with a compressed gas (non-limiting examples of compressed gases include, air, nitrogen, or argon). Multiple passes or applications may be required to obtain the desired coating layer thickness or the desired mass on the substrate.

The solvents are removed to form the coating layer. Some residual solvent will typically remain in the coating layer. The solvents are removed by evaporation and evaporation may be enhanced by methods such as passing a stream of air over the device during or after the application of the coating solution.

With regard to the substrate, that is the device to which a coating is applied, a portion of the medical device or the whole device itself can be formed of material containing a biodegradable polymer, a biostable polymer, or a combination thereof. In some embodiments the device may be formed essentially of a polymer. The device may be made of a metal or metal alloy, a ceramic, or a glass. Any combination of a metal, a metal alloy, a ceramic, a glass, a biostable polymer and a biodegradable polymer may be used for constructing the medical device, or a portion of the medical device.

The coating having a hygroscopic layer over an implantable device such as the balloon of a balloon catheter may have a range of thickness. The coating may be of substantially uniform thickness. In certain embodiments, an individual coating layer, or a coating including more than one layer, over at least a portion of an implantable device may have a thickness of less than or equal to about 30 micron, less than or equal to about 20 micron, less than or equal to about 10 micron, or less than or equal to about 5 micron.

Non-limiting examples of polymers, which may or may not be the hygroscopic polymers defined above, that can be used to fabricate a device whether as part of the body of the device itself or as the a polymer in any of the coating layers on the device, include, without limitation, poly(N-acetylglucosamine) (Chitin), Chitosan, poly(hydroxyvalerate), poly(lactide-co-glycolide), poly(hydroxybutyrate), poly(hydroxybutyrate-co-valerate), polyorthoester, polyanhydride, poly(glycolic acid), poly(glycolide), poly(L-lactic acid), poly(L-lactide), poly(D,L-lactic acid), poly(L-lactide-co-glycolide), poly(D,L-lactide), poly(caprolactone), poly(trimethylene carbonate), polyethylene amide, polyethylene acrylate, poly(glycolic acid-co-trimethylene carbonate), co-poly(ether-esters) (e.g., PEO/PLA), polyphosphazenes, biomolecules (e.g., fibrin, fibrinogen, cellulose, starch, collagen and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (e.g., polyvinyl chloride), polyvinyl ethers (e.g., polyvinyl methyl ether), polyvinylidene halides (e.g., polyvinylidene chloride), polyacrylonitrile, polyvinyl ketones, polyvinyl aromatics (e.g., polystyrene), polyvinyl esters (e.g., polyvinyl acetate), acrylonitrile-styrene copolymers, ABS resins, polyamides (e.g., Nylon 66 and polycaprolactam), polycarbonates, polyoxymethylenes, polyimides, polyethers, polyurethanes, rayon, rayon-triacetate, cellulose and derivates thereof (e.g., cellulose acetate, cellulose butyrate, cellulose acetate butyrate, cellophane, cellulose nitrate, cellulose propionate, cellulose ethers, and carboxymethyl cellulose), ethylene vinyl alcohol copolymer (commonly known by the generic name EVOH or by the trade name EVAL®), poly(butyl methacrylate), poly(vinylidene fluoride-co-hexafluoropropylene) (e.g., SOLEF® 21508, available from Solvay Solexis PVDF of Thorofare, N.J.), polyvinylidene fluoride (otherwise known as KYNAR®, available from Atofina Chemicals of Philadelphia, Pa.), poly(tetrafluoroethylene-co-hexafluoropropylene-co-vinylidene fluoride), ethylene-vinyl acetate copolymers, polyethylene glycol. and copolymers thereof.

### Method of Treating or Preventing Disorders

A medical device with a coating according to one of the embodiments of the present invention can be used to treat, prevent or diagnose various conditions or disorders.

In one embodiment of the method, the medical device is formed of a material including and/or includes a coating containing at least one biologically active agent selected from paclitaxel, docetaxel, estradiol, nitric oxide donors, super oxide dismutases, super oxide dismutase mimics, 4-amino-2,2,6,6-tetramethylpiperidine-1-oxyl (4-amino-TEMPO), tacrolimus, dexamethasone, dexamethasone-acetate, rapamycin, rapamycin derivatives, 40-O-(2-hydroxy)ethyl-rapamycin (everolimus), 40-O-(2-ethoxy)ethyl-rapamycin (biolimus), 40-O-(3-hydroxy)propyl-rapamycin, 40-O-[2-(2-hydroxy)ethoxy]ethyl-rapamycin, 40-O-tetrazole-rapamycin, 40-epi-(N-1-tetrazolyl)-rapamycin (zotarolimus), novolimus, myolimus, pimecrolimus, imatinib mesylate, midostaurin, clobetasol, progenitor cell-capturing antibodies, prohealing drugs, cRGD, prodrugs thereof, co-drugs thereof, and any combinations thereof.

In certain embodiments, optionally in combination with one or more other embodiments described herein, the device used in the method of treatment is selected from stents, grafts, stent-grafts, catheters, leads and electrodes, clips, shunts, closure devices, valves, and balloon catheters. In a specific embodiment, the implantable device is a balloon catheter.

While particular embodiments of the present invention have been shown and described, it will be obvious to those skilled in the art that changes and modifications can be made without departing from this invention in its broader aspects. Therefore, the claims are to encompass within their scope all such changes and modifications as fall within the true sprit and scope of this invention.

What is claimed is:

1. A balloon catheter, the balloon comprising a coating free of substances having a molecular weight of greater than 40,000 Daltons, the coating comprising a hygroscopic layer comprising a hygroscopic polymer disposed over a drug layer, the drug layer comprising at least 40% by weight drug.

2. The device of claim 1, wherein 80% or more by weight of the hygroscopic layer dissolves in about 8 minutes or fewer.

3. The device of claim 1, wherein the hygroscopic layer consists essentially of one or more hygroscopic polymers and one or more drugs.

4. The device of claim 1, wherein the hygroscopic layer consists essentially of polyvinylpyrrolidone and a plasticizer.

5. The device of claim 1, wherein the drug layer comprises at least 55% by weight of drug.

6. The device of claim 1, wherein the drug is selected from the group consisting of paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl (4-amino-TEMPO), biolimus, tacrolimus, dexamethasone, dexamethasone-acetate, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, 40-O-tetrazole-rapamycin, 40-epi-(N-1-tetrazolyl)-rapamycin (ABT-578), temsirolimus, deforolimus, novolimus, myolimus, γ-hirudin, clobetasol, pimecrolimus, imatinib mesylate, midostaurin, cRGD, prodrugs thereof, co-drugs thereof, and combinations thereof.

7. The implantable device of claim 1, wherein the hygroscopic polymer is an amphiphilic polymer, a dissolvable polymer, or any combination thereof.

8. The device of claim 1, wherein the hygroscopic polymer is selected from the group consisting of polyvinylpyrrolidone (PVP), poly(carboxymethyl cellulose) (poly(CMC)), poly[N-(2-hydroxypropyl)methacrylamide] (poly(HPMA)), poly(ethylene glycol), and combinations thereof.

9. The device of claim 7, wherein the amphiphilic polymer is a hydrophobic polymer with hygroscopic pendant groups covalently attached.

10. The device of claim 9, wherein the hygroscopic pendant groups are selected from the group consisting of choline, phosphoryl choline, ammonium groups, sulfuric acid groups, sulfonic acid groups, and combinations thereof.

11. The device of claim 7, wherein the amphiphilic polymer is selected from the group consisting of poly(n-butyl methacrylate-phosphorylcholine) (PBMA-PC), poly(ester amide)-phosphorylcholine (PEA-PC), polylactide-phosphorylcholine (PLA-PC), polylactide-phosphorylcholine (PLA-PC), polyethylene glycol-poly(caprolactone)-di- or tri-blocks (PEG-PCL), polyethylene glycol-polylactide di- or tri-blocks (PEG-PLA), polyethylene glycol-poly(lactide-glycolide) di- or tri-blocks (PEG-PLGA), poly(ethylene oxide)-b-poly(propylene oxide)-b-poly(ethylene oxide) block copolymers, and combinations thereof.

12. The device of claim 7, wherein the dissolvable polymer is selected from the group consisting of polyvinyl alcohol (PVA); poly n-butyl methacrylate-phosphorylcholine (PBMA-PC); poly(ester amide)-phosphorylcholine (PEA-PC); polylactide-phosphorylcholine (PLA-PC); poly(lactic acid-co-glycolic acid)-phosphorylcholine (PLGA-PC); silk-elastin, dynamers, or tri-blocks such as poly(caprolactone)-polyethylene glycol-poly(caprolactone) (PCL-PEG-PCL); (PLA-PEG-PLA); poly(lactide-glycolide)-polyethylene glycol-poly(lactide-glycolide) tri-blocks (PLGA-PEG-PLGA); and combinations thereof.

13. The device of claim 1, wherein the drug layer comprises a hygroscopic polymer.

14. The device of claim 13, wherein the hygroscopic polymer of the drug layer is selected from the group consisting of polyvinylpyrrolidone (PVP); poly(carboxymethyl cellulose) (poly(CMC)), and poly[N-(2-hydroxypropyl)methacrylamide] (poly(HPMA)), poly(ethylene glycol) and combinations thereof.

15. The device of claim 13, wherein the hydroscopic polymer of the drug layer is an amphiphilic polymer comprising a hydrophobic polymer comprising hygroscopic pendant groups.

16. The device of claim 15, wherein the hygroscopic group of the drug layer is selected from the group consisting of choline, phosphoryl choline, ammonium groups, sulfuric acid groups, sulfonic acid groups, and combinations thereof.

17. The device of claim 13, wherein the hydroscopic polymer of the drug layer is an amphiphilic polymer selected from the group consisting of poly(n-butyl methacrylate-phosphorylcholine) (PBMA-PC), poly(ester amide)-phosphorylcholine (PEA-PC), polylactide-phosphorylcholine (PLA-PC), polylactide-phosphorylcholine (PLA-PC), polyethylene glycol-poly(caprolactone)-di- or tri-blocks (PEG-PCL), polyethylene glycol-polylactide di- or tri-blocks (PEG-PLA), polyethylene glycol-poly(lactide-glycolide) di- or tri-blocks (PEG-PLGA), poly(ethylene oxide)-b-poly(propylene oxide)-b-poly(ethylene oxide), and combinations thereof.

18. The device of claim 13, wherein the hydroscopic polymer of the drug layer is a dissolvable polymer selected from the group consisting of polyvinyl alcohol (PVA); poly n-butyl methacrylate-phosphorylcholine (PBMA-PC); poly(ester amide)-phosphorylcholine (PEA-PC); polylactide-phosphorylcholine (PLA-PC); poly(lactic acid-co-glycolic acid)-phosphorylcholine (PLGA-PC); silk-elastin, dynamers, or tri-blocks such as poly(caprolactone)-polyethylene glycol-poly(caprolactone) (PCL-PEG-PCL); (PLA-PEG-PLA); poly(lactide-glycolide)-polyethylene glycol-poly(lactide-glycolide) tri-blocks (PLGA-PEG-PLGA), and combinations thereof.

19. The device of claim 1, wherein the coating is capable of releasing up to about 50% to about 100% of the drug in the drug layer within about 5 to 60 minutes upon exposure to the aqueous environment.

20. A method for minimizing loss of a drug in an implantable device comprising a drug coated balloon, comprising forming a hygroscopic layer comprising a hygroscopic polymer over the balloon, the balloon comprising a layer comprising a drug;
wherein the hygroscopic layer has a water absorbing capacity of between about 20 wt % and about 500 wt % of the dry weight of the hygroscopic layer within about 5 to 60 minutes upon exposure to an aqueous environment.
